(19) United States
(12) Patent Application Publication
(10) Pub. No.: US 2014/0356349 A1
(43) Pub. Date: Dec. 4, 2014

(54) BIOMARKERS OF RESISTANCE TO HER2 INHIBITORS

(71) Applicants: Seoul National University R&DB Foundation, Seoul (KR); Industry-University Cooperation Foundation Hanyang University, Seoul (KR)

(72) Inventors: In-Cheol SHIN, Seoul (KR); Tae-Hoon LEE, Gyeongbuk-do (KR); Ji-Hyun JU, Seoul (KR); Kyung-Min LEE, Seoul (KR); Won-Seok YANG, Seoul (KR); Dong-Young NOH, Seoul (KR)

(21) Appl. No.: 13/908,382

(22) Filed: Jun. 3, 2013

(51) Int. Cl. A61K 39/395; G01N 33/574; C12Q 1/68

(52) U.S. Cl. CPC A61K 39/39558; C12Q 1/6886; G01N 33/57488
USPC 424/133.1; 435/6.12; 436/501; 435/7.92

(57) ABSTRACT

Provided is a method of suppressing resistance to an anticancer drug, comprising administering an inhibitor of expression or activity of ECM 1 (Extracellular Matrix Protein 1) to a cancer cell or an individual with cancer.

### BACKGROUND

The present disclosure relates to biomarkers capable of diagnosing resistance or tolerance to HER2 (human epidermal growth factor receptor 2)-targeted therapeutics, and more particularly, to markers capable of diagnosing resistance to HER2 inhibitors, for example, Herceptin (trastuzumab), and treatment of breast cancer and vaccines for preventing a recurrence using the same.

Breast cancer (BCa) is the most common cancer among women, and the second leading cause of cancer death in women (Ries L A G, et al. (eds). SEER Cancer Statistics Review, 1975-2003, National Cancer Institute, Bethesda, Md.). Major advance in breast cancer treatment over the past 20 years was a significant improvement in disease-free survival (DFS). For example, some therapies with the use of antibodies reactive with tumor-associated antigens slowed disease progression and other therapies blocked certain cell growth processes to prevent a disease recurrence. Despite recent advances in breast cancer treatment, a substantial number of patients will eventually die from their recurred disease. Thus, there is a need for treatments that prevent or delay or inhibit recurrent disease progression.

Targeted passive immunotherapy based on HER2/neu proto-oncogene has been focused mainly on the use of Tz (trastuzumab, product name: Herceptin). Tz is a recombinant humanized monoclonal antibody that binds to an extracellular juxtamembrane domain of HER2/neu protein. Tz has been approved by regulatory authorities, and is prescribed for treating HER2/neu overexpressing (IHC 3+ or FISH >2.0) tumors in specified adjuvants for metastatic breast cancer patients and node-positive breast cancer patients. Tz passed a number of clinical tests and is now conventionally used for treatment of metastatic patients and for adjuvant therapy in patients with high-risk breast cancer that overexpresses HER2/neu.

However, Tz shows limited activity in patients with low to middle levels of HER2/neu expression. Accordingly, based on the previous results shown with Tz, it is not anticipated that immunogenic peptide vaccines that target HER2/neu will be effective in cancer patients with lower and middle levels of HER2/neu tumor expression.

Particularly, due to tolerance (resistance) to Tz (Herceptin), clinical signs, radiologic signs and symptoms are not often completely disappeared, and tumors often relapse (recur).

To overcome adverse effects, metastasis, and resistance, which are limitations of conventional anticancer drugs, researches that use “molecular biology” and genetic information of “genome project” are anticipated to accelerate in the future research and development of anticancer drugs.

In particular, since resistance to anticancer drugs often occurs during cancer treatment, a complete cure is known to be difficult. Thus, for cancer treatment, how to control resistance due to use of anticancer drugs and the solution for a case in which it is incapable of using anticancer drugs from the beginning due to resistance to anticancer drugs are as important as using anticancer drugs for primary removal of cancer. The present inventors studied mechanism of resistance to anticancer drugs, methods to overcome resistance, and suitable materials to overcome resistance. These are necessary to conquer cancer, and a shortcut to an ultimate goal, cancer conquest.

Accordingly, in the technical field, there is a need for developing diagnostic markers of resistance and vaccines, which would provide a reliable protection against disease recurrence for breast cancer patients with resistance to anticancer drugs, who are in a clinically relaxed state.

Thus, the present inventors analyzed Erk, Akt, HER2 activities and proteome (secretory proteome) for cell groups that showed resistance when Herceptin (trastuzumab) was used as a HER2 (human epidermal growth factor receptor 2)-targeting therapeutic agent, discovered proteins which show the potential as a biomarker of resistance to Herceptin, and completed the present invention.

### SUMMARY

The present disclosure provides biomarker compositions for diagnosing resistance to anticancer drugs, particularly, HER2 inhibitors, and methods of using the same.

The present disclosure also provides kits for diagnosing resistance to HER2 inhibitors.

The present disclosure also provides methods of suppressing resistance to HER2 inhibitors.

### Technical Solution

In accordance with one aspect of the present invention, a method of suppressing drug resistance in a patient suffering cancer resistant to an anticancer drug, comprising: administering an inhibitor of expression or activity of ECM 1 (Extracellular Matrix Protein 1) to the patient is provided.

The anticancer drug may be an HER2 inhibitor and may be Herceptin (trastuzumab).

The cancer may be selected from the group consisting of ovarian cancer, peritoneal cancer, fallopian tubal cancer, breast cancer, nonsmall cell lung cancer (NSCLC), squamous cell carcinoma, prostate cancer, and colorectal cancer.

The inhibitor of expression or activity of ECM 1(Extracellular Matrix Protein 1) may be any one selected from the group consisting of antisense oligonucleotides, short interfering RNAs, short hairpin RNAs, and RNAis, which bind complementarily to mRNA of ECM 1 gene, or may be any one selected from the group consisting of compounds, peptides, peptide mimetics, and antibodies, which bind complementarily to ECM 1 protein.

The ECM 1 (Extracellular Matrix Protein 1) may be a polypeptide having nucleotide of SEQ ID NO: 1.

In accordance with another aspect of the present invention, a method for determining prognosis of a cancer patient, comprising: measuring an amount of ECM 1 (Extracellular Matrix Protein 1) expression from a body fluid of the cancer patient to whom an anticancer drug was administered is provided.

The method may further comprise determining that the cancer patient has poor prognosis when the amount of ECM 1 is significantly higher than normal level.

The anticancer drug may be an HER2 inhibitor and may be Herceptin (trastuzumab).

The cancer may be selected from the group consisting of ovarian cancer, peritoneal cancer, fallopian tubal cancer, breast cancer, nonsmall cell lung cancer (NSCLC), squamous cell carcinoma, prostate cancer, and colorectal cancer.

The ECM 1 (Extracellular Matrix Protein 1) may a polypeptide having nucleotide of SEQ ID NO: 1.

In a preferred embodiment of the present invention, secretome analysis, RT-PCR and Western blotting revealed that ECM 1 mRNA or protein expression in Herceptin-resistant cell is significantly higher than BT474 wild type cell (control). ECM 1 mRNA expression in BT474 Herceptin-resistant clone (HR-22) is much higher (about 5-15 fold) than in BT474 wild type cell, which did not show Herceptin resistance (see FIG. 6). Therefore, expression or amount of ECM 1 of a cancer patient could be a critical factor to determine the patient's prognosis.

The method allows for determination of prognosis of the cancer patient and provision of information for additional treatment, by determining therapeutic effect of the anticancer drug through measurement of the amount of ECM 1 expression to enable a decision on whether resistance to the anticancer drug is present or not.

In accordance with another aspect of the present invention, a method for predicting drug resistance of a patient suffering cancer, comprising measuring an amount of ECM 1 (Extracellular Matrix Protein 1) expression from a body fluid of the patient to whom an anticancer drug was administered is provided.

The method may further comprise determining that the cancer patient has poor prognosis when the amount of ECM 1 is significantly higher than normal level.

The anticancer drug may be an HER2 inhibitor and may be Herceptin (trastuzumab).

The cancer may be selected from the group consisting of ovarian cancer, peritoneal cancer, fallopian tubal cancer, breast cancer, nonsmall cell lung cancer (NSCLC), squamous cell carcinoma, prostate cancer, and colorectal cancer.

The ECM 1 (Extracellular Matrix Protein 1) may a polypeptide having nucleotide of SEQ ID NO: 1.

### Advantageous Effects

The present invention enables to decide whether resistance to HER2 inhibitors for anticancer treatment, preferably to Herceptin (trastuzumab), is present or not, by measuring ECM 1 (Extracellular Matrix Protein 1) expression, and enables to use appropriate alternative anticancer drugs, and establish new models for anticancer drug development to overcome resistance to anticancer drugs.

Moreover, the present invention can be applied as a testing method for determining metastatis of cancer cells by measuring ECM 1 (Extracellular Matrix Protein 1) expression, and can be widely used for development of inhibitors of resistance to anticancer agents, preventive agents, or therapeutic agents for tumors, in combination with conventional tumor markers.

### BRIEF DESCRIPTION OF THE DRAWINGS

Exemplary embodiments can be understood in more detail from the following description taken in conjunction with the accompanying drawings, in which:

FIG. 1 is a photograph about the process of establishing Herceptin-resistance (HR) clones;

FIG. 2 is the result of a MTT assay using HR clones;

FIG. 3 is the results of a 3D-growth assay for examining effects of Herceptin on proliferation in HR clones;

FIG. 4 is the results of proteome analysis of BT474 Herceptin-resistant (tolerant) cells using LC-MS/MS;

FIG. 5 is the results of Secretome analysis using LC-MS/MS; and

FIG. 6 is the results of RT-PCR and Western blotting for a discovered Herceptin-resistant marker.

### DETAILED DESCRIPTION OF EMBODIMENTS

Definition of the terms used in the present invention is as follows.

“Subject” or “patient” refers to any single individual in need of treatment, including humans, cows, dogs, guinea pigs, rabbits, chicken, insects, etc. In addition, any subject, who does not exhibit any clinical findings of diseases and participated in clinical trials, or subjects who participated in epidemiological research, or subjects who were used as a control are included in the subject. The subject of one embodiment of the present invention was humans.

“Tissue or cell sample” refers to a similar cell aggregate obtained from a tissue of a subject or patient. A source of the tissue or cell sample may be a fresh, frozen and/or conserved organ or tissue sample or a solid tissue from biopsy or aspirate; blood or any blood cell; a cell of any point of pregnancy or development of a subject. The tissue sample may also be a primary or cultured cell, or cell line. Tissue or cell samples obtained from primary or metastatic tumor were used in one embodiment of the present invention. The tissue sample may include compounds which are not intermixed with the tissue in nature such as preservatives, anticoagulants, buffers, fixatives, nutrients, antibiotics, or the like. For the purposes of the present invention, a “section” of the tissue sample may be a single part or piece of the tissue sample, e.g. a thin slice of tissue or cells cut from the tissue sample. It would be understood that multiple sections of tissue samples may be taken and subjected to analysis according to the present invention, provided that it would be understood that the present invention includes a methodology whereby the same section of tissue sample is analyzed at both morphological and molecular levels, or is analyzed with respect to both protein and nucleic acid.

“Marker” refers to a nucleotide sequence or encoded product thereof (e.g., a protein) used as a point of reference when identifying a locus or linked locus. A marker may be derived from genomic nucleotide sequence or from expressed nucleotide sequences (e.g., from RNA, nRNA, mRNA, cDNA, and the like), or from an encoded polypeptide. The term may include nucleic acid sequences complementary to or flanking the marker sequences, such as nucleic acids used as probes or primer pairs capable of amplifying the marker sequence.

By “nucleic acid” is meant to include any DNA or RNA, for example, chromosomal, mitochondrial, viral and/or bacterial nucleic acid present in tissue sample. “Nucleic acid” may encompass either or both strands of a double stranded nucleic acid molecule and include any fragment or portion of an intact nucleic acid molecule.

By “gene” is meant any nucleic acid sequence or portion thereof with a functional role in encoding or transcribing a protein or regulating other gene expression. The gene may consist of all the nucleic acids responsible for encoding a functional protein or only a portion of the nucleic acids responsible for encoding or expressing a protein. The nucleic acid sequence may contain a genetic abnormality within exons, introns, initiation or termination regions, promoter sequences, other regulatory sequences or unique sequences adjacent to the gene.

“Antibody” is used in the broadest sense, and specifically may include intact monoclonal antibodies, polyclonal antibodies, multi-specific antibodies (for example, bispecific antibodies) formed from at least two intact antibodies, and antibody fragments having biological activity of interest.

“Label” refers to a compound or composition which is conjugated, or fused and conjugated directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable.

“Cancer”, “tumor”, or “malignant” refer to or describe the physiological condition in mammals that is typically characterized by unregulated cell growth. Examples of cancers in the present invention are those that express HER2, and include, but are not limited to, ovarian cancer, peritoneal cancer, fallopian tubal cancer, breast cancer, nonsmall cell lung cancer (NSCLC), squamous cell carcinoma, prostate cancer, or colorectal cancer.

“Inhibitor” refers to a substance which inhibits, blocks, or reduces expression or activity of a specific gene. An inhibitor of HER2 and an inhibitor of ECM 1 used in the present invention are substances which inhibit, block, or reduce expression or activity of HER2 and ECM 1 (Extracellular Matrix Protein 1), respectively. Activation mechanism of an inhibitor is not particularly limited. Examples of inhibitors may include organic or inorganic compounds, polymeric compounds such as proteins, carbohydrates, lipids, composites of various compounds.

“Treatment” refers to an approach to obtain beneficial or desirable clinical outcomes. For the purposes of the present invention, beneficial or desirable outcomes include, but are not limited to, alleviation of symptoms, diminishment of extent of disease, stabilized (e.g., not worsening) state of disease, delay or slowing of disease progression, amelioration or temporary palliation of the disease state, and remission (whether partial or total), whether detectable or undetectable. In addition, “treatment” may mean prolonging survival as compared to expected survival if not receiving treatment. “Treatment” refers to both therapeutic treatment and preventive or prophylactic measures. The treatments include treatments required for already occurred disorders as well as disorders to be prevented. “Palliating” diseases means diminishing extent of disease state and/or undesirable clinical symptoms and/or slowing down or extending time course of progression as compared to the untreated case.

“About” means an amount, level, value, number, frequency, percent, dimension, size, weight or length changed by 30, 25, 20, 25, 10, 9, 8, 7, 6, 5, 4, 3, 2 or 1% from a reference amount, level, value, number, frequency, percent, dimension, size, weight or length.

Unless otherwise required, through the present disclosure, the terms “include” and “including” include a suggested step or element, or a group of steps or elements, but it should be understood not to exclude any other step or element, or group of steps or elements.

Herein after, the present invention is described in detail.

The present invention relates to resistance to anticancer drugs, and in particular, to resistance to inhibitors of (anticancer) HER2, and most preferably, to biomarkers capable of diagnosing resistance (tolerance) to Herceptin (trastuzumab) which is a Her2 (ErbB2) antibody.

“Resistance” to an anticancer drug means that cancer cells cause genetic changes in response to the anticancer drug, and thereby they avoid an attack of the anticancer drug and weaken effects of anticancer treatment. The term “resistance” can be used in the same meaning as “tolerance”.

HER2 (ErbB2) gene encodes a transmembrane glycoprotein of 185,000 daltons in molecular weight belonging to erbB family of epithelial growth factor receptors. Ligand binding induces the formation of erbB-homo- and erbB-heterodimers leading to activation of cytoplasmic kinase region. HER2 is a receptor having no ligand, and a preferred partner of heterodimerization among ligand-binding EGFR family, EGFR/erbB1, HER3/erbB3 and HER4/erbB4. As a co-receptor, HER2 mediates signal transduction and leads to mitogenesis, apoptosis, angiogenesis, and cell differentiation. Application of any changes to a strictly controlled erbB receptor which signalizes pathways causes to significant abnormalities and tumor formation. HER2 gene is amplified and overexpressed in about 20-30% of invasive breast carcinomas and is related to increased metastatic, latent, and poor prognosis. In addition, the overexpression of HER2 receptor occurs in a variety of human cancers including uterus, prostate, stomach, lung, bladder, and kidney carcinomas.

An inhibitor of HER2 means a substance that inhibits (suppresses) the expression or activity of HER2 (ErbB2) gene. For example, the inhibitor of HER2 expression may be any one selected from the group consisting of antisense oligonucleotides, short interfering RNAs, short hairpin RNAs, and RNAis, which bind complementarily to mRNA of HER2 gene, or may be any one selected from the group consisting of compounds, peptides, peptide mimetics, and antibodies, which bind complementarily to HER2 protein. Most preferably, the inhibitor of HER2 expression is Herceptin (trastuzumab), which is known as a HER2 antibody.

Herceptin (trastuzumab) is a humanized monoclonal antibody which blocks HER2 (human epidermal growth factor receptor 2). Overexpression of HER2 which is also known as ErbB2 is observed in 20-30% of breast cancer patients and is related to invasive breast cancers and patients show a poor prognosis. Herceptin is used for patients with metastatic breast cancer having tumors that overexpress HER2. However, the overall response rate was below 35% and what decides the response to Herceptin has not been known yet.

In addition, patients with PTEN-deficient breast cancers had significantly resistant responses to Herceptin (chemotherapy combination) than those with normal PTEN. Additionally, it was found that compounds which inhibit PI3K rescued PTEN loss-induced Herceptin resistance, suggesting that PI3K inhibitors could overcome this resistance.

In particular, it was found that Herceptin treatment quickly increased PTEN membrane localization from cytoplasm and PTEN phosphatase activity by reducing PTEN tyrosine phosphorylation via Src inhibition, and reducing PTEN in breast cancer cells by PTEN gene inactivation conferred Herceptin resistance.

Thus, the present inventors analyzed Erk, Akt, HER2 activities and proteomes in cells of which sensitivity was reduced in response to anticancer action of the HER2 inhibitor Herceptin (trastuzumab) of breast cancer tissues in breast cancer patients, and thereby, found that Herceptin (trastuzumab) resistance (tolerance) was associated with the overexpression of ECM 1 (Extracellular Matrix Protein 1).

Accordingly, in accordance with one aspect of the present invention, biomarkers for diagnosing anticancer drug resistance or biomarker compositions including the same are provided. The anticancer drug may be a therapeutic agent targeting HER2, and an inhibitor of HER2 is preferable, and the most preferable example may be Herceptin (trastuzumab).

The cancer may be a cancer which expresses HER2. For example, the cancer may be selected from the group consisting of ovarian cancer, peritoneal cancer, fallopian tubal cancer, breast cancer, nonsmall cell lung cancer (NSCLC), squamous cell carcinoma, prostate cancer, and colorectal cancer. Most preferably, the cancer is a breast cancer.

The biomarker ECM 1 (Extracellular Matrix Protein 1) of resistance to the anticancer drug may be ECM 1, and particularly, is characterized by including a polypeptide having nucleotide of SEQ ID NO: 1.

In addition, the present invention uses a fact that the ECM 1 expression is increased in a cancer cell which expresses resistance to an inhibitor of HER2, such as Herceptin (trastuzumab), and thus, provides a method of diagnosing resistance to an anticancer drug, particularly an inhibitor of HER2, such as Herceptin (trastuzumab) in early stage by examining the overexpression of ECM 1 in a cancer cell of a patient that expresses HER2.

The present invention also relates to a method of using ECM 1 (Extracellular Matrix) to increase sensitivity in response to anticancer action of Herceptin (trastuzumab), the method including suppressing the expression of ECM 1 (Extracellular Matrix Protein 1).

The method of suppressing the expression of ECM 1 may use any one selected from the group consisting of antisense oligonucleotides, short interfering RNAs, short hairpin RNAs, and RNAis, which bind complementarily to mRNA of ECM 1 gene, or may use any one selected from the group consisting of compounds, peptides, peptide mimetics, and antibodies, which bind complementarily to ECM 1 protein.

“RNAi” refers to RNA interference. RNA interference is a phenomenon of specific gene silencing, which is well conserved in most organisms. It is considered to be a kind of gene surveillance mechanism that cells use to defend against viral infection or inhibit transposons or remove abnormal mRNAs. Particularly, a phenomenon of gene silencing by small RNA is referred to as RNA interference in a broad sense, and RNA interference in a narrow sense refers to a phenomenon of mRNA degradation by siRNA. In addition, RNA interference refers to a gene silencing experiment technique using siRNA. “small RNA” refers to a ribonucleic acid of 17-25 nucleotides in length functioning to regulate gene expression in vivo. Small RNA is classified largely into microRNA (shorted to miRNA) and small interfering RNA (shortened to siRNA) depending on its generating mode. miRNA is generated from a partially double stranded RNA (hairpin RNA) and siRNA is derived from long double stranded RNA (dsRNA). To define generally, small RNA which plays an important role in various regulation processes in a living body is a microRNA, and small RNA which is used to experimental technologically regulate the expression of a specific gene is an siRNA. miRNA is produced naturally within cells and binds specifically to a specific mRNA and inhibits protein synthesis from mRNA. siRNA is a small RNA to be introduced artificially into cells and plays a role in binding to a specific mRNA having a complementary sequence and degrading the mRNA.

The term “siRNA” refers to a double stranded DNA molecule which prevents the translation of a target mRNA. A standard technique of introducing siRNA into a cell, including DNA as a template from RNA is transcribed is used. The siRNA may be either dsRNA or shRNA. “dsRNA” refers to a two RNA molecules construct consisting of a single strand and other strand having a complementary sequence to the single strand, and two molecules have complementary sequences and thus, combine together to form a double-stranded RNA molecule. Double stranded nucleic acid sequences may include “sense” or “antisense” sequences of RNA selected from protein coding sequences of a heterologous gene sequence, and RNA molecules selected from non-coding regions of the target gene. The term “shRNA” refers to a siRNA having a stem-loop structure, including first and second regions which are complementary each other, i.e., sense and antisense strands. If the degree of complementarity and space of the complementary region are sufficient, bindings of sufficient base pairs between the regions occurs, and first and second regions are connected by the loop region, and the loop region is made through the absence of base pairs between nucleic acids (or nucleic acid analogs). The loop region of the shRNA is a single strand region between sense and antisense strands, and may be also referred to as an“intervened single strand.” Preferable example of the present invention may be a siRNA of ECM 1.

“Antibody” is used in the broadest sense, and specifically includes intact monoclonal antibodies, polyclonal antibodies, multi-specific antibodies (for example, bispecific antibodies) formed from at least two intact antibodies, and antibody fragments as long as they have biological activity of interest.

In accordance with another aspect of the present invention, anticancer compositions including inhibitors of expression or activity of ECM 1 are provided.

As described above, the inhibitor of expression or activity of ECM 1 may be, but is not limited to, any one selected from the group consisting of antisense oligonucleotides, short interfering RNAs, short hairpin RNAs, and RNAis, which bind complementarily to mRNA of ECM 1 gene, or any one selected from the group consisting of compounds, peptides, peptide mimetics, and antibodies, which bind complementarily to ECM 1 protein.

This inhibitor of ECM 1 expression overcomes (suppresses) effectively resistance to Herceptin (trastuzumab) anticancer drug, and induces growth inhibition and apoptosis of cancer cells, and thus, can be used effectively as an active ingredient for anticancer resistant therapeutic agents.

The anticancer composition of the present invention may further contain one or more types of other active ingredients showing an identical or similar function in addition to the active ingredient.

The anticancer composition of the present invention may be prepared further including one or more types of a pharmaceutically acceptable carrier in addition to the active ingredient described above. As the pharmaceutically acceptable carrier, saline solution, sterilized water, linger's solution, buffer saline, dextrose solution, maltodextrin solution, glycerol, ethanol, liposome, and at least one combination thereof, may be used, and if necessary, other typical additives such as antioxidants, buffer solution, bacteriostatic agents, etc., may be added. Moreover, it may be formulated in the form of an injectable formulation such as aqueous solution, suspension and emulsion, a pill, a capsule, a granule, or a tablet by supplementarily adding diluents, dispersing agents, surfactants, binders and lubricants. And it may be used combining a target organ-specific antibody or other ligands with the carrier to act specifically upon a target organ. Furthermore, it may be preferably formulized according to each disease or ingredients using a suitable method in the art, for example, a method disclosed in Remington's Pharmaceutical Science (the latest edition), Mack Publishing Company, Easton Pa.

The present invention also provides a method of suppressing resistance of an inhibitor of HER2, and in particular, preferably, resistance to Herceptin (trastuzumab), in HER2-expressing cancers, the method including administering a pharmaceutically effective amount of an inhibitor of ECM 1 expression to an individual.

The administration method may be, but is not particularly limited to, a parenteral administration (for example, intravenous, subcutaneous, intraperitoneal, or topical application) or oral administration depending on a method of interest. Although it is preferable to administer parenterally, more preferable to inject intravenously, the present invention is not limited thereto.

The range of dosage varies according to a patient's body weight, age, sex, health status, diet, administration time, administration method, excretion rate, the severity of disease, etc. The daily dosage for a compound is in the range of about 0.1 to 100 mg/kg, preferably 0.5 to 10 mg/kg. It is preferable to administer the inhibitor of ECM 1 expression one or more times a day, however, the present invention is not limited thereto.

Meanwhile, in the present invention, the rate of apoptosis of cancer cells can be increased by suppression of ECM 1 expression and sensitivity to anticancer action can be increased. Thus, the present invention provides a new method of overcoming resistance to an inhibitor of HER2 and a material for the same.

The present invention also relates to a method of using ECM 1 for discovery of new genes that induce resistance (tolerance) an inhibitor of HER2, such as Herceptin (trastuzumab). Thus, by using the method, the present invention provides a new diagnosis method and a treatment method, for overcoming resistance by identifying new genes involved in resistance to Herceptin (trastuzumab), the anticancer drug.

Accordingly, in accordance with a similar aspect of the present invention, a method for determining prognosis of a cancer patient and providing information for treatment including measuring an amount of ECM 1 expression from a body fluid of the cancer patient to whom an anticancer drug was administered is provided. The anticancer drug may be an inhibitor of HER2, and may be Herceptin (trastuzumab).

In the method of the present invention, detection of ECM 1 expression level statistically significantly higher than a normal range tells that the patient has resistance to the anticancer drug, for example, the inhibitor of HER2. In a sample of the individual who has been being administered the inhibitor of HER2, detection of ECM 1 expression level in the normal range means a success of the cancer therapy, and detection of ECM 1 expression level higher than the normal range in the sample means a gain of resistance to the inhibitor of HER2 that has been being administered. Furthermore, in the sample, detection of normal ECM 1 expression level in the normal range means the prognosis of the administered patient is good; however, detection of ECM 1 expression level higher than the normal range in the sample means that resistance to the inhibitor of HER2 occurred and the prognosis of the patient is not good.

Expression of a biomarker ECM 1 in a sample may be analyzed by a number of methodologies, many of which are known in the art and understood by those skilled in the art, including but not limited to, immunohistochemical and/or Western analysis, quantitative blood based assays (for example, serum ELISA) (to examine levels of protein expression), biochemical enzymatic activity assays, in situ hybridization, Northern blot analysis and/or PCR analysis of mRNAs, and genome Western blot analysis (to examine, for example, gene deletion or amplification), as well as any one of the wide variety of assays that can be performed by gene and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Meanwhile, the present invention provides a method of using ECM 1 as a label substance to develop an enhancer of anticancer drug sensitivity, capable of reacting more sensitively to an inhibitor of HER2, for example, Herceptin (trastuzumab) anticancer drug.

Also, the present invention relates to a method of using ECM 1 as a label marker to decrease adverse effects which may be generated when used in a higher dose and increase anticancer effects by suppressing ECM 1 expression and thus, reducing the use of HER2 inhibitors.

In accordance with another aspect of the present invention, a kit for diagnosing resistance to anticancer drugs including a biomarker of resistance to HER2 inhibitors or an antibody which binds specifically to an immunogenic fragment thereof is provided.

The anticancer drug is a therapeutic agent targeting HER2, and may be an inhibitor of HER2, and most preferably, Herceptin (trastuzumab) may be used. The cancer may be a cancer which expresses HER2. For example, the cancer may be selected from the group consisting of ovarian cancer, peritoneal cancer, fallopian tubal cancer, breast cancer, nonsmall cell lung cancer (NSCLC), squamous cell carcinoma, prostate cancer, and colorectal cancer. Most preferably, the cancer is a breast cancer.

The kit for diagnosing resistance of the present invention may additionally include one or more substances which are reactive with ECM 1, and a reagent for detecting reaction products and instructions related thereto. For example, one or more substances which are reactive with ECM 1 may be an RNA or DNA complementary to RNA or DNA of ECM 1, and an antibody which binds to ECM 1 protein, and the reagent for detecting reaction products may be a nucleic acid or protein marker and a color developing reagent.

For example, in the case that the kit is applied for PCR amplification process, the kit of the present invention may optionally include reagents required for PCR amplification, for example, buffer solution, DNA polymerases (for example, heat-stable DNA polymerases obtained from Thermus aquatics (Taq), Thermus thermophilus (Tth), Thermus filiformis (Tfi), Thermis flavus (Tfi), Thermococcus literalis (Tli) or Pyrococcus furiosus (Pfu)), DNA polymerase cofactors, and dNTPs. In the case that the kit of the present invention is applied for immunoassay, the kit of the present invention may optionally include secondary antibodies and a substrate of a label. The kit of the present invention may be manufactured in a number of separate packages or compartments including the above reagent components.

Accordingly, for example, the kit for diagnosing resistance to HER2 inhibitors may include additionally a secondary antibody conjugate to which a label body that develops a color in response to the reaction with a substrate is conjugated; a color-developing substrate solution to be reacted with the label body; a washing solution; and an enzymatic reaction stop solution. The label body of the secondary antibody conjugate may be selected from the group consisting of horseradish peroxidase (HRP), alkaline phosphatase, colloidal gold, fluorescein and dyes.

Like this, the biomarker for diagnosing resistance to HER2 inhibitors and the kit for diagnosing resistance to HER2 inhibitors of cancer patients in accordance with the present invention enables to predict resistance to HER2 inhibitors, e.g., Herceptin (trastuzumab) of HER2-expressing cancer patients in an early stage, and allows for selective and individual anticancer drug treatment for HER2-expressing patients.

### EXAMPLES

Hereinafter, the present invention will be described in more detail by way of examples. The examples are provided for illustrative purposes only and thus, it would be obvious to those skilled in the art that the scope of the present invention is not construed to be limited by the examples.

### Example 1

### Establishment of Herceptin Resistant (HR) Clones

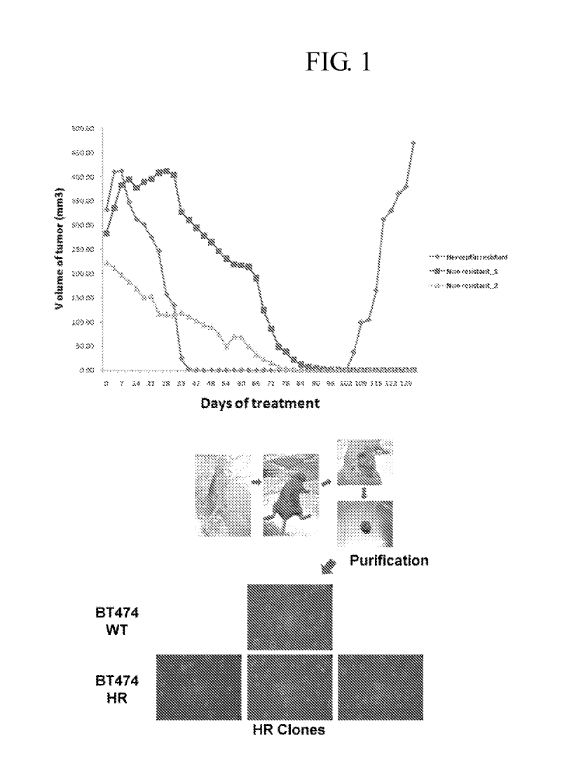

In order to perform a xenograft of HER2 protein-positive human breast cancer cell BT474 to BALB/C nude immunodeficient mice (CAnN.Cg-Foxn1nu/CrljOri, Orient), 60 day release 17-beta-estradiol pellet (Innovative research, 0.72 mg) was transplanted in advance. The next day, 2×10⁷ BT474 (ATCC) cells prepared in PBS were injected in the flank of a mouse via 22-guage needle (Christoph A. Ritter et al. 2007. Clin Cancer Res). Once the transplanted BT474 cells developed to tumors of a size about 250 mm³ (width×length/2), mice were treated with a final administration concentration of 20 mg/kg (based on a mouse weight) Herceptin (Roche, USA) twice a week. Since actual mice ranged from 20 to 25 g in weight, about 400-500 μg per mouse was administered via IP route. When the size of the tumors was reduced by continuous administration of Herceptin, and then the tumors were recurred under Herceptin administration status, those tumors were regarded as BT474 cells with Herceptin resistance, harvested, digested with IMEM (0.25% Trypsin, Gibco), and cultured. Individuals in which the tumor size was decreased were classified into non-resistant-1 and non-resistant-2 individuals, and individuals in which the tumor was decreased and recurred were classified as Herceptin-resistant individuals. Then, tumors in the Herceptin resistant individuals were harvested, and BT-474 HR (Herceptin resistance) clones were established through a primary culture (see FIG. 1).

### Example 2

### MTT Assay

A MTT assay was performed using HR clones (HR-4, HR-8, HR-14, HR-15, HR-16, HR-17, HR-22) established in Example 1.

5×10³ cells were seeded into a 96-well plate. After 24 hours, cells were treated with 20 μg/ml of Herceptin. After 48 hours, cells were allowed to react with a MTT solution for 3 hours. Formed formazan crystals were dissolved with DMSO (Sigma) and O.D value was measured by using ELISA. For a control, cells treated with the same amount of PBS solution were used.

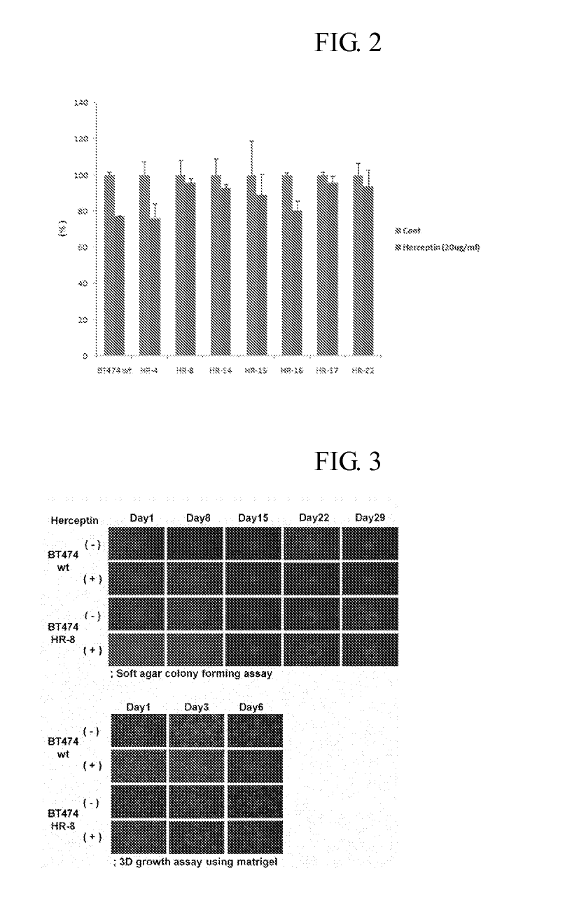

Consequently, as shown in FIG. 2, HR clones showed resistance to Herceptin. The results demonstrated that among BALB/C nude immunodeficient mice (CAnN.Cg-Foxn1nu/CrljOri, Orient) transplanted with HER2 protein-positive human breast cancer cell BT474 in Example 1, HR clones actually have resistance to Herceptin.

### Example 3

### 3D-Growth Assay

Effects of herceptin on proliferation of HR clones harvested from BALB/C nude immunodeficient mice (CAnN.Cg-Foxn1nu/CrljOri, Orient) transplanted with HER2 protein-positive human breast cancer cell BT474 in Example 1 were examined by performing a 3D-growth assay.

A bottom layer of 0.8% low melting point agarose(Sigma) was made into a 6-well plate, and when the bottom layer became solid, 3×10⁴ cells of BT474 wild type (wt) or HR clone cells (HR-8) were admixed to 0.4% agarose to make a cell suspension layer (0.4% agarose cell layer). 20 μg/ml of Herceptin was treated to the cell suspension layer and cultured. The same concentration of Herceptin was additionally treated every 72 hours. The time of cell seeding into the 6-well plate was set to day 0, and on 1^{st}, 8^{th}, 15^{th}, 22^{nd}, and 29^{th} day, cells were observed by using an optical microscope at a magnification of 100×.

Consequently, as shown in FIG. 3, unlike BT474 wild type cells, changes in cell viability caused by Herceptin treatment were not observed in the HR clone (HR-8) group (upper panel).

In addition, 5×10⁵ cells of BT474 wild type or HR clone cells (HR-8) were cultured in a 6-well plate in a three-dimensional environment made up of media:Matrigel (BD Biosciences)=3:1 and resistance of HR clone to Herceptin was examined. 20 μg/ml of Herceptin was treated to the 6-well plate and cultured. Herceptin was additionally treated every 72 hours and cells were observed at a magnification of 100×.

Consequently, as shown in FIG. 3, BT474 herceptin resistance cells were found out (lower panel).

### Example 4

### Protein Analysis

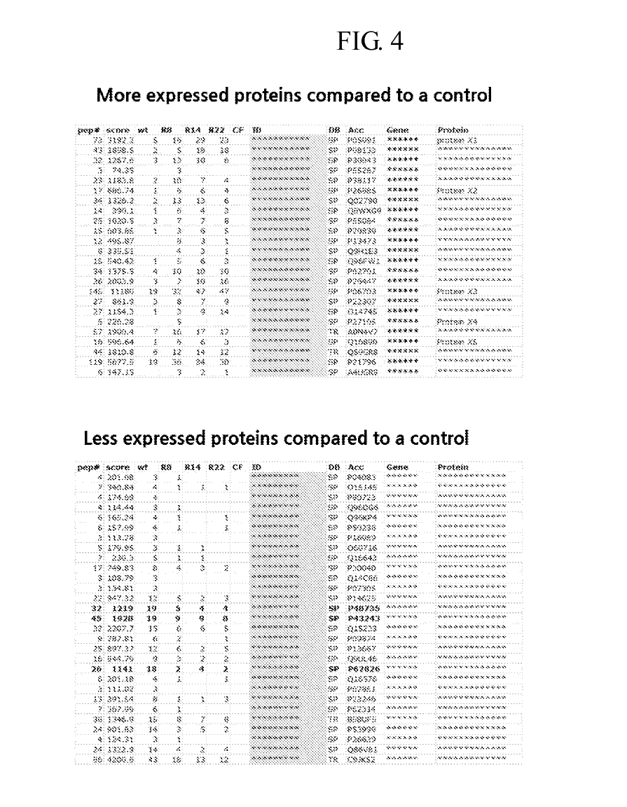

Proteomes of BT474 and HR clones (HR-8, HR-22) of which resistance to Herceptin was confirmed in Examples 2 and 3 were analyzed (differential proteome profiling by LC-MS/MS) and a number of proteins that show the potential as biomarkers of resistance to Herceptin were discovered (FIG. 4).

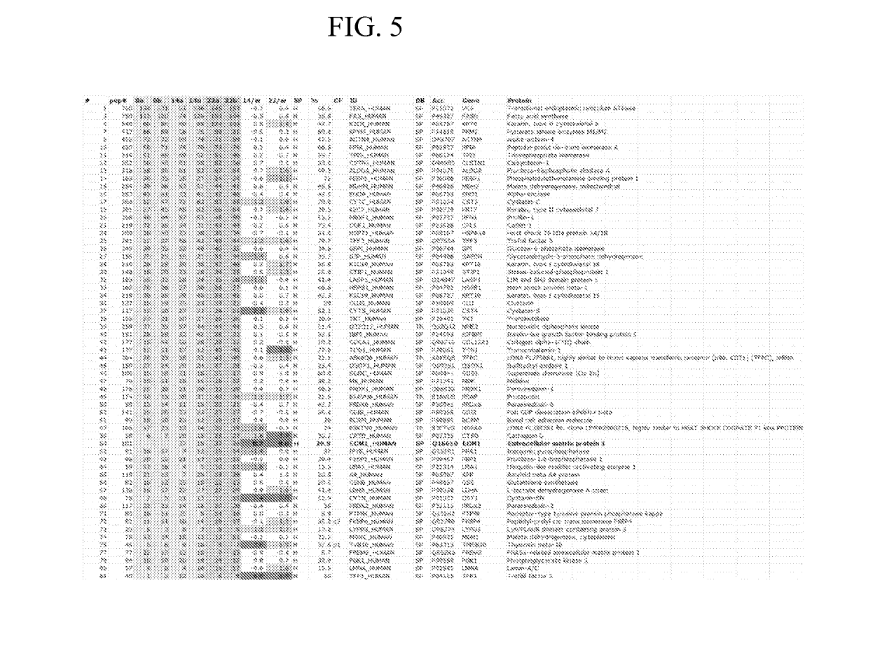

In addition, secretomes of media obtained from BT474 HR clones (HR-8, HR-22) of which resistance to Herceptin was confirmed in Examples 2 and 3 were analyzed by LC-MS/MS method (FIG. 5).

From those results, a biomarker ECM 1 of resistance (tolerance) to Herceptin was discovered. It is represented by a nucleotide sequence of SEQ ID NO: 1.

### Example 5

### RT-PCR and Western Blotting

BT474 wildtype and HR clone cells (HR-22) were obtained and cells were lysed with Trizol for 5 minutes. Chloroform (Sigma) was added and centrifugation was carried out. RNA in the supernatant was precipitated with isopropyl alcohol and the obtained RNA was used for cDNA synthesis by RT-PCR. The following ECM 1-specific primers were prepared and PCR was carried out (94° C. for 2 min; 29 cycles of 94° C. for 25 sec, 60° C. for 20 sec, and 72° C. for 40 sec; and 72° C. for 5 min).

Forward-
(SEQ ID NO: 2)
5′ AGG CTC GGT TCT CCT GCT TCC AG 3′
Reverse-
(SEQ ID NO: 3)
5′ TTG GGG TAA GGA GCC CGA CGG 3′

PCR products were examined by 1% agarose gel electrophoresis.

BT474 wildtype and HR clone cells were obtained and lysed with a RIPA lysis buffer for 15 min. 30 μg of lysate was electrophoresed on 10% SDS-gel and transferred onto a nitrocellulose membrane. Blocking was carried out with 5% skim milk. Proteins were cultured with a primary antibody (ECM 1, Actin-Santacruz) for overnight, cultured with a secondary antibody for 2 hours, and allowed to react with ECL solution to develop a film. In order to detect Protein E in the media, the ratio of TCA (sigma):media was made to be 1:4. Proteins were precipitated and centrifuged. Pellets were washed with acetone and lysed with a lysis buffer for 1 hour to obtain lysates. Western blotting was carried out by the same method described above.

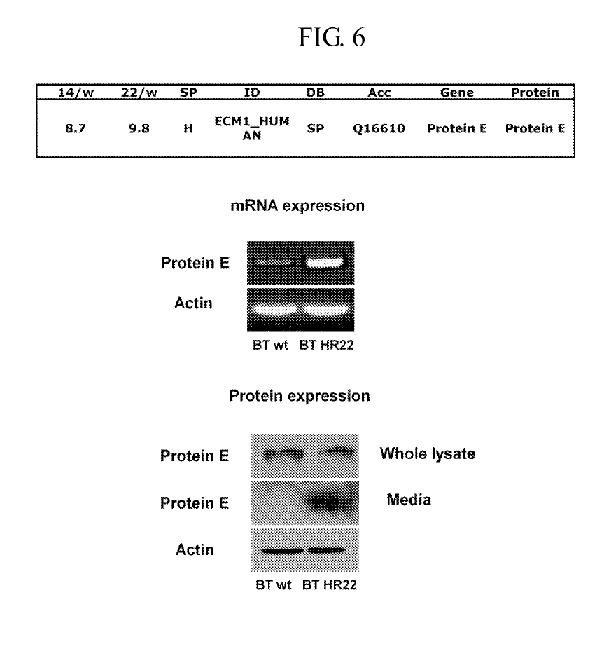

The results are shown in FIG. 6. While the expression of ECM 1 was high at the transcriptional level, it was not high at the intracellular protein level. In addition, the amount released out of cells was far greater in the HR cells than in the BT474 wildtype cells. That is, the results showed that ECM 1 (Extracellular Matrix Protein 1) could be used effectively as an extracellular Herceptin resistance marker.

Although the biomarkers of resistance to HER2 inhibitors have been described with reference to the specific embodiments, they are not limited thereto. Therefore, it will be readily understood by those skilled in the art that various modifications and changes can be made thereto without departing from the spirit and scope of the present invention defined by the appended claims.

What is claimed is:

1. A method of suppressing drug resistance in a patient suffering cancer resistant to an anticancer drug, comprising: administering an inhibitor of expression or activity of ECM 1(Extracellular Matrix Protein 1) to the patient.

2. The method of claim 1, wherein the anticancer drug is an HER2 inhibitor.

3. The method of claim 2, wherein the HER2 inhibitor is Herceptin (trastuzumab).

4. The method of claim 1, wherein the cancer is selected from the group consisting of ovarian cancer, peritoneal cancer, fallopian tubal cancer, breast cancer, nonsmall cell lung cancer (NSCLC), squamous cell carcinoma, prostate cancer, and colorectal cancer.

5. The method of claim 1, wherein the inhibitor of expression or activity of ECM 1 is any one selected from the group consisting of antisense oligonucleotides, short interfering RNAs, short hairpin RNAs, and RNAis, which bind complementarily to mRNA of ECM 1 gene, or any one selected from the group consisting of compounds, peptides, peptide mimetics, and antibodies, which bind complementarily to ECM 1 protein.

6. The method of claim 1, wherein the ECM 1 is a polypeptide having nucleotide of SEQ ID NO: 1.

7. A method for determining prognosis of a cancer patient, comprising: measuring an amount of ECM 1 (Extracellular Matrix Protein 1) expression from a body fluid of the cancer patient to whom an anticancer drug was administered.

8. The method of claim 7, further comprising determining that the cancer patient has poor prognosis when the amount of ECM 1 is significantly higher than normal level.

9. The method of claim 7, wherein the anticancer drug is an HER2 inhibitor.

10. The method of claim 9, wherein the HER2 inhibitor is Herceptin.

11. The method of claim 8, wherein the ECM 1 is a polypeptide having nucleotide of SEQ ID NO: 1.

12. The method of claim 8, wherein the cancer is selected from the group consisting of ovarian cancer, peritoneal cancer, fallopian tubal cancer, breast cancer, nonsmall cell lung cancer (NSCLC), squamous cell carcinoma, prostate cancer, and colorectal cancer.

13. A method for predicting drug resistance of a patient suffering cancer, comprising measuring an amount of ECM 1 (Extracellular Matrix Protein 1) expression from a body fluid of the patient to whom an anticancer drug was administered.

14. The method of claim 13, further comprising predicting that the patient has drug resistance when the amount of ECM 1 is significantly higher than normal level.

15. The method of claim 13, wherein the anticancer drug is an HER2 inhibitor.

16. The method of claim 13, wherein the HER2 inhibitor is Herceptin (trastuzumab).

17. The method of claim 13, wherein the cancer is selected from the group consisting of ovarian cancer, peritoneal cancer, fallopian tubal cancer, breast cancer, nonsmall cell lung cancer (NSCLC), squamous cell carcinoma, prostate cancer, and colorectal cancer.

18. The method of claim 13, wherein the ECM 1 is a polypeptide having nucleotide of SEQ ID NO: 1.


SEQUENCE LISTING (3 sequences)

SEQ ID NO: 1 - protein, 567 aa, Homo sapiens
MGTTARAALVLTYLAVASAASEGGFTATGQRQLRPEHFQEVGYAAPPSPPLSRSLPMDHPDSSQHGPPFEGQSGKEGRGPRPHSQPWLGERVGCSHIPPSIVQPPPSQEATPLQQEKLLPAQLPAEKEVGPPLPQEAVPLQKELPSLQHPNEQKEGTPAPFGDQSHPEPESWNAAQHCQQDRSQGGWGHRLDGFPPGRPSPDNLNQICLPNRQHVVYGPWNLPQSSYSHLTRQGETLNFLEIGYSRCCHCRSHTNRLECAKLVWEEAMSRFCEAEFSVKTRPHWCCTRQGEARFSCFQEEAPQPHYQLRACPSHQPDISSGLELPFPPGVPTLDNIKNICHLRRFRSVPRNLPATDPLQRELLALIQLEREFQRCCRQGNNHTCTWKAWEDTLDKYCDREYAVKTHHHLCCRHPPSPTRDECFARRAPYPNYDRDILTIDIGRVTPNLMGHLCGNQRVLTKHKHIPGLIHNMTARCCDLPFPEQACCAEEEKLTFINDLCGPRRNIWRDPALCCYLSPGDEQVNCFNINYLRNVALVSGDTENAKGQGEQGSTGGTNISSTSEPKEE

SEQ ID NO: 2 - DNA, 23 bp, Artificial Sequence
  Forward primer
aggctcggttctcctgcttccag

SEQ ID NO: 3 - DNA, 21 bp, Artificial Sequence
  Reverse primer
ttggggtaaggagcccgacgg